(19) United States
(12) Patent Application Publication
(10) Pub. No.: US 2023/0158089 A1
(43) Pub. Date: May 25, 2023

(54) PROBIOTIC COMPOSITIONS AND METHODS OF USE

(71) Applicant: CHANDLER BIOPHARMACEUTICAL CORPORATION, Milton (CA)

(72) Inventor: Richmond A. CHANDLER, Milton (CA)

(21) Appl. No.: 17/917,725

(22) PCT Filed: Apr. 9, 2021
(86) PCT No.: PCT/CA2021/050477

§ 371 Date: Oct. 7, 2022

Related U.S. Application Data
(60) Provisional application No. 63/007,370, filed on Apr. 9, 2020.

(51) Int. Cl. A61K 35/747; A61K 35/744; A61K 9/00; A61P 31/14

(52) U.S. Cl. CPC A61K 35/747; A61K 35/744; A61K 9/0053; A61P 31/14

(57) ABSTRACT

Probiotic compositions capable of generating a high CFU count and methods to improve the health of a subject including to treat or prevent a viral infection are disclosed.

### CROSS-REFERENCE TO RELATED APPLICATIONS

This application claims the benefit of U.S. Provisional Application No. 63/007,370, filed on Apr. 9, 2020, the contents of which are incorporated herein by their entirety.

### FIELD OF THE INVENTION

The invention relates to a method of treatment and prevention of viral infection. Specifically, the invention provides a method of treatment or prevention of a viral infection including COVID-19 by administering probiotics to a subject.

### BACKGROUND OF THE DISCLOSURE

Probiotic bacteria are known to stimulate the immune system. Given the COVID-19 pandemic and the various mutant strains, there is a need to provide a means of boosting the immune system to prevent viral infection.

### SUMMARY OF THE DISCLOSURE

Provided herein are methods of treating or preventing a viral infection in a subject by administering a probiotic composition to the subject.

In some embodiments, the method comprises administering a probiotic composition in an effective amount to boost a subject's immune system and prevent a SARS-CoV-2 viral infection or reduce its severity.

In some embodiments, the method comprises administering a probiotic composition in an effect amount to provide an anti-viral effect to treat a subject diagnosed with COVID-19.

In some embodiments, a kit comprises containers including a probiotic composition is provided.

### DETAILED DESCRIPTION

Disclosed herein are methods of treating or preventing a viral infection by administering an effective amount of a probiotic composition. Further, the probiotic compositions disclosed herein have been found to be advantageous because in described combinations, the probiotic bacteria within the probiotic composition are capable of producing CFUs in the trillions. It is generally understood that the more CFUs available in a probiotic, the more effective the probiotic composition will be at boosting the immune system, supporting the gut health, supporting the limbic system, and providing antibiotic and antiviral effects. The benefits of probiotics are continually being studied; however, the ability of certain combinations of probiotic bacteria strains to produce substantially high CFUs is not known in the art. Finally, kits are described for packaging and transporting the disclosed probiotic compositions.

### Ranges and Definitions

Unless defined otherwise herein, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used herein and in the appended claims, the singular forms “a”, “an”, and “the” include plural referents unless the context clearly dictates otherwise. It is noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “and/or” and “any combination thereof” and their grammatical equivalents may be used interchangeably. Solely for illustrative purposes, the following phrases “A, B, and/or C” or “A, B, C, or any combination thereof” can mean “A individually; B individually; C individually; A and B; B and C; A and C; and A, B, and C.”

As used herein, the term “about” in relation to a reference numerical value and its grammatical equivalents includes the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.” For example, the phrase “about 45 mM, 40 mM, 35 mM,” and so on means “about 45 mM, about 40 mM, about 35 mM,” and so on.

As used herein, the term “active” or “activity” for the purposes herein refers to a biological activity of a native or naturally-occurring probiotic molecule, wherein “biological” activity refers to a biological function (either inhibitory or stimulatory) caused by a native or naturally-occurring probiotic molecule.

Thus, “biologically active” or “biological activity” when used in conjunction with the probiotic molecules described herein refers to probiotic molecule or amino acid sequence that exhibits or shares an effector function of the native probiotic molecule or sequence. For example, the probiotic molecules described herein have the biological activity of preventing, inhibiting, or treating an enteric viral infection in an animal.

By “probiotic” it is generally defined as a live microbial food supplement which beneficially affects the host human or animal by improving its intestinal microbial balance.

According to an aspect, isolated probiotic bacteria are provided, the probiotic bacteria being effective in vitro and in vivo to prevent and/or treat viral infection.

“Substantially free” herein means less than about 5%, typically less than about 2%, more typically less than about 1%, even more typically less than about 0.5%, most typically less than about 0.1% contamination with other bacteria, yeast, fungi, or mold.

As used herein, “treatment” or “therapy” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of cellular degeneration or damage, such as due to viral infection, or may render the cells more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to prevent or treat a viral infection. Effective amounts of the agents described herein may vary according to factors such as the disease state, age, sex, and weight of the subject. Dosage or treatment regimens may be adjusted to provide the optimum therapeutic response, as is understood by a skilled person.

Moreover, a treatment regime of a subject with a therapeutically effective amount may consist of a single administration, or alternatively comprise a series of applications. The length of the treatment period depends on a variety of factors, such as the severity of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The agents of the present invention may, in aspects, be administered before, during or after treatment with conventional therapies for the disease or disorder in question, such as a viral infection.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

The term CFUs meaning colony forming units is used to describe the amount of probiotic bacteria in the probiotic composition at about time 0 (To) or soon after production of the product. CFUs can also refer to the amount of CFUs listed on packaging of a probiotic composition. Finally, CFUs may refer to the amount of CFUs in the probiotic composition at the end of a 2-year shelf-life. The amount of CFUs may be estimated using a standard algorithm known to those in the industry that produce probiotic compositions.

Administration “in combination with” one or more further therapeutic agents includes simultaneous (concurrent) and consecutive administration in any order.

“Carriers” as used herein include pharmaceutically acceptable carriers, excipients, or stabilizers that are nontoxic to the cell or mammal being exposed thereto at the dosages and concentrations employed. Often the pharmaceutically acceptable carrier is an aqueous pH buffered solution. Examples of pharmacologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, and dextrins; chelating agents such as EDTA; sugar alcohols such as mannitol and sorbitol; salt-forming counterions such as sodium; and/or nonionic surfactants such as TWEEN™m, polyethylene glycol (PEG), and PLURONICS™.

A “liposome” is a small vesicle composed of various types of lipids, phospholipids, and/or surfactant which is useful for delivery of a drug to a subject, such as a mammal. The components of the liposome are commonly arranged in a bilayer formation, similar to the lipid arrangement of biological membranes.

As will be apparent to those of skill in the art upon reading this disclosure, each of the individual embodiments described and illustrated herein has discrete components and features, which can be readily separated from or combined with the features of any of the other several cases without departing from the scope or spirit of the present invention. Any recited method can be carried out in the order of events recited or in any other order that is logically possible.

### Probiotics

In an illustrative embodiment, a probiotic is provided to a subject that utilizes a probiotic composition to boost the subject's immune response to a viral infection. In some embodiments, the probiotic composition comprises biologically active probiotic bacteria. In some embodiments, the probiotic composition comprises isolated probiotic bacteria strains substantially free of contamination. In some embodiments, the probiotic composition is formulated for ingestion by the subject.

In some embodiments, the probiotic composition comprises at least one strain of probiotic bacteria to at least six strains of probiotic bacteria. In some embodiments, the probiotic composition comprises at least one strain or up to six strains of probiotic bacteria. In some embodiments, the probiotic composition comprises between three and five strains, between four and six strains, or between three and six strains of probiotic bacteria. In some embodiments, the probiotic composition comprises a single strain of probiotic bacteria. In some embodiments, the probiotic composition comprises two strains of probiotic bacteria. In some embodiments, the probiotic composition comprises three strains of probiotic bacteria. In some embodiments, the probiotic composition comprises four strains of probiotic bacteria. In some embodiments, the probiotic composition comprises five strains of probiotic bacteria. In some embodiments, the probiotic composition comprises six strains of probiotic bacteria. In some embodiments, wherein the probiotic composition comprises two strains A and B, the two strains may be present in the probiotic composition at a ratio of about 10:1, 9:1, 8:1, 7:1, 6:1, 5:1, 4:1, 3:2, 3:1, 2:1, 1:1, 1:2, 2:3, 1:3, 1:4, 1:5, 1:6, 1:7, 1:8, 1:9, or 1:10.

The following embodiments, include the percentage of strains at the end of product production and/or at the end of a 2-year shelf-life. In some embodiments, wherein the probiotic composition comprises three strains A, B, and C, the three strains may be present in an amount where strain A is present in an amount between about 1% to about 98%, strain B is present in an amount of about 1% to about 98%, and strain C is present in an amount of about 1% to about 98%. For example, strain A may be present at about 90%, strain B may be present at about 5%, and strain C may be present at about 5%. In another example, strain A may be present at about 33%, strain B is present at about 33%, and strain C is present at about 33%. Alternatively, strain A may be present at about 10%, strain B may be present at about 15%, and strain C may be present at about 7% of the probiotic composition, and wherein the remaining 68% of the probiotic composition comprises carriers, filler, favors, vitamins, fiber or a combination thereof.

In some embodiments, wherein the probiotic composition comprises four strains A, B, C, and D, the four strains may be present in an amount where strain A is present at an amount of about 1% to about 97%, strain B may be present in an amount of about 1% to about 97%, strain C may be present in an amount of about 1% to about 97%, and strain D may be present in an amount of about 1% to about 97%. For example, strain A may be present at about 1%, strain B may be present about 1%, strain C may be present at about 97%, and strain D may be present at about 1%. In another example, strain A may be present at about 25%, strain B may be present at about 25%, strain C may be present at about 25%, and strain D may be present at about 25%. Alternatively, strain A may be present at about 48%, strain B may be present at about 12%, strain C may be present at about 27%, and strain D may be present at about 3% of the probiotic composition, and wherein the remaining 10% of the probiotic composition comprises carriers, filler, favors, vitamins, fiber or a combination thereof.

In some embodiments, wherein the probiotic composition comprises five strains A, B, C, D, and E, the five strains may be present in an amount where strain A is present at an amount of about 1% to about 96%, strain B is present in an amount of about 1% to about 96%, strain C is present in an amount of about 1% to 96%, strain D is present in an amount of about 1% to about 96%, and strain E is present in an amount of about 1% to about 96%. For example, strain A may be present at about 1%, strain B may be present at about 2%, strain C may be present at about 3%, strain D may be present at about 4%, and strain E may be present at about 90%. Alternatively, strain A may be present at about 20%, strain B may be present at about 20%, strain C may be present at about 20%, strain D may be present at about 20%, and strain E may be present at about 20%. Alternatively, strain A may be present at 8%, strain B may be present at 27% strain C may be present at 19%, strain D may be present at 10%, and strain E may be present at 5%, and wherein the remaining 31% of the probiotic composition comprises carriers, filler, favors, vitamins, fiber or a combination thereof.

In some embodiments, wherein the probiotic composition comprises six strains A, B, C, D, E, and F, the six strains may be present in an amount where strain A is present at about 1% to about 95%, strain B is present at about 1% to about 95%, strain C is present about 1% to about 95%, strain D is present at about 1% to about 95%, strain E is present at about 1% to about 95%, and strain F is present at about 1% to about 95%.

In some embodiments, the probiotic composition comprises Streptococcus, Lactobacillus, Lactococcus, or a combination thereof. In some embodiments, the probiotic composition comprises Streptococcus, Lactobacillus, and Lactococcus. In some embodiments, the probiotic composition consists essentially of Streptococcus, Lactobacillus, and Lactococcus.

In some embodiments, the probiotic composition comprises a strain of Streptococcus. In some embodiments, the probiotic composition comprises Streptococcus salivarius thermophiles.

In some embodiments, the probiotic composition comprises a strain of Streptococcus present in the probiotic composition between about 0.001% to about 100%, about 0.1% to about 99.9%, or about 1% to about 99% by weight of the total probiotic composition. In some embodiments, the strain of Streptococcus is present in an amount of about of about 1%, 5%, 10%, 15%, 20%, 25%, 30%, 35%, 40%, 45%, 50%, 55%, 60%, 65%, 70%, 75%, 80%, 85%, 90%, 95%, or 99%. In certain aspects the strain of Streptococcus is substantially free of contaminates.

In some embodiments, the probiotic composition comprises a range of about 100 million to about 600 billion colony forming units (CFUs) of a Streptococcus strain per gram of probiotic composition. In some embodiments, the Streptococcus strain is present at about 100 million CFUs to about 500 billion CFUs, about 200 million CFUs to about 100 billion CFUS, or about 500 million CFUS to about 100 billion CFUs per gram of probiotic composition. In some embodiments, the probiotic composition comprises about 100 million, 200 million, 300 million, 400 million, 500 million, 600 million, 700 million, 800 million, 900 million, 1 billion, 10 billion, 20 billion, 30 billion, 40 billion, 50 billion, 60 billion, 70 billion, 80 billion 90 billion, 100 billion, 200 billion, 300 billion, 400 billion, 500 billion, 600 billion, 1 trillion, 1.25 trillion, 1.50 trillion, 1.75 trillion, 2 trillion, 2.25 trillion 2.5 trillion, 2.75 trillion, or 3 trillion CFUs of the Streptococcus strain per gram of probiotic composition.

In some embodiments, the probiotic composition comprises a strain of Lactobacillus. In some embodiments, the Lactobacillus strain is selected from the group Lactobacillus acidophilus, Lactobacillus rhamnosus, and Lactobacillus delbrueckii. In some embodiments, the Lactobacillus strain includes delbrueckii and rhamnosus. In some embodiments, wherein the Lactobacillus comprises two strains, the strains may be present at a ratio of about 10:1, 9:1, 8:1, 7:1, 6:1, 5:1, 4:1, 3:2, 3:1, 2:1, 1:1, 1:2, 2:3, 1:3, 1:4, 1:5, 1:6, 1:7, 1:8, 1:9, or 1:10. In some embodiments, the Lactobacillus comprises three strains.

In some embodiments, the Lactobacillus strains includes acidophilus, delbrueckii, and rhamnosus. In some embodiments, wherein the Lactobacillus comprises three strains, the first Lactobacillus strain may be present between about 1% to about 98% of the three Lactobacillus strains, the second Lactobacillus strain may be present between about 1% to about 98% of the three Lactobacillus strains, and the third strain of Lactobacillus may be present between about 1% to about 98% of the three Lactobacillus strains. In some embodiments, the probiotic composition comprises a Lactobacillus comprising acidophilus, delbrueckii, and rhamosus.

In some embodiments, the Lactobacillus is present in the probiotic composition between about 0.001% to about 100%, about 0.1% to about 99.9%, or about 1% to about 99% by weight of the total probiotic composition. In some embodiments, the Lactobacillus is present in an amount of about of about 1%, 5%, 10%, 15%, 20%, 25%, 30%, 35%, 40%, 45%, 50%, 55%, 60%, 65%, 70%, 75%, 80%, 85%, 90%, 95%, or 99%. In certain aspects the Lactobacillus is substantially free of contaminates.

In some embodiments, the probiotic composition comprises a range of about 100 million to about 600 billion colony forming units (CFUs) of Lactobacillus per gram of probiotic composition. In some embodiments, the Lactobacillus is present at about 100 million CFUs to about 500 billion CFUs, about 200 million CFUs to about 100 billion CFUS, or about 500 million CFUS to about 100 billion CFUs per gram of probiotic composition. In some embodiments, the probiotic composition comprises about 100 million, 200 million, 300 million, 400 million, 500 million, 600 million, 700 million, 800 million, 900 million, 1 billion, 10 billion, 20 billion, 30 billion, 40 billion, 50 billion, 60 billion, 70 billion, 80 billion 90 billion, 100 billion, 200 billion, 300 billion, 400 billion, 500 billion, 600 billion, 700 billion, 800 billion, 900 billion, 1 trillion, 1.25 trillion, 1.50 trillion, 1.75 trillion, 2 trillion, 2.25 trillion 2.5 trillion, 2.75 trillion, or 3 trillion CFUs of the Lactobacillus strain per gram of probiotic composition.

In some embodiments, the probiotic composition comprises Lactococcus. In some embodiments, the Lactococcus includes lactis. In some embodiments, the probiotic composition comprises Lactococcus lactis.

In some embodiments, the probiotic composition comprises Lactococcus present in the probiotic composition between about 0.001% to about 100%, about 0.1% to about 99.9%, or about 1% to about 99% by weight of the total probiotic composition. In some embodiments, the strain of Lactococcus is present in an amount of about of about 1%, 5%, 10%, 15%, 20%, 25%, 30%, 35%, 40%, 45%, 50%, 55%, 60%, 65%, 70%, 75%, 80%, 85%, 90%, 95%, or 99%. In certain aspects the strain of Lactococcus is substantially free of contaminates.

In some embodiments, the probiotic composition comprises a range of about 100 million to about 600 billion colony forming units (CFUs) of Lactococcus per gram of probiotic composition. In some embodiments, the Lactococcus is present at about 100 million CFUs to about 500 billion CFUs, about 200 million CFUs to about 100 billion CFUS, or about 500 million CFUS to about 100 billion CFUs per gram of probiotic composition. In some embodiments, the probiotic composition comprises about 100 million, 200 million, 300 million, 400 million, 500 million, 600 million, 700 million, 800 million, 900 million, 1 billion, 10 billion, 20 billion, 30 billion, 40 billion, 50 billion, 60 billion, 70 billion, 80 billion 90 billion, 100 billion, 200 billion, 300 billion, 400 billion, 500 billion, 600 billion, 1 trillion, 1.25 trillion, 1.50 trillion, 1.75 trillion, 2 trillion, 2.25 trillion 2.5 trillion, 2.75 trillion, or 3 trillion CFUs of the Lactococcus per gram of probiotic composition.

In an illustrative embodiment, the probiotic composition comprises Streptococcus, Lactobacillus, and Lactococcus prepared in a form for digestion in a subject's gut. In some embodiments, the probiotic composition comprises Streptococcus salivrius thermophilus, Lactobacillus, and Lactoccocus. In some embodiments, the probiotic composition comprises Streptococcus, Lactobacillus selected from delbrueckii, rhamnosus, acidophilus, or a combination thereof, and Lactococcus. In some embodiments, the probiotic composition comprises Streptococcus, Lactobacillus, and Lactoccocus lactis.

In some embodiments, the probiotic composition comprises Streptococcus salivrius thermophilus, Lactobacillus including delbrueckii, rhamnosus, acidophilus, and Lactococcus lactis.

In some embodiments, the probiotic composition comprises Streptococcus salivrius thermophilus, Lactobacillus including delbrueckii and rhamnosus, and Lactococcus lactis. In some embodiments, the probiotic composition comprises between about 1 trillion CFUs to about 2 trillion CFUs/g of probiotic composition. In some embodiments, the probiotic composition comprises about 1.25 trillion CFUs to about 1.50 trillion CFUs/g of probiotic composition. In some embodiments, the probiotic composition comprises about 1.3 trillion CFUs/g of probiotic composition.

In some embodiments, the probiotic composition comprises Streptococcus and Lactobacillus. In some embodiments, the Lactobacillus comprises delbrueckii and rhamnosus. In some embodiments, the Lactobacillus delbrueckii comprises the subspecies bulgaricus. In some embodiments, the probiotic composition comprises Streptococcus thermophilus, Lactobacillus delbrueckii subs. bulgaricus, and Lactobacillus rhamnosus.

The exemplified strains herewith are identified by genotype and phenotype, characterized and identified by genome sequencing and duly deposited in the International Depositary Authority of Canada (IDAC) located at National Microbiology Laboratory, Public Health Agency of Canada, 1015 Arlington Street, Winnipeg, Manitoba Canada R3E 3R2These strains have been deposited in the IDAC on Dec. 10, 2019, and have been assigned the following identifiers: Streptococcus thermophilus CIB8002/IDAC 101219-05; Lactobacillus delbrueckii subs. bulgaricus CBC8000/IDAC 101219-01; Lactobacillus rhamnosus CBC8001/IDAC 101219-03; and Lactobacillus acidophilus CBC8004/IDAC 101219-02.

In some embodiments, the probiotic composition comprises between about 100 million CFUs to about 5 trillion CFUs, about 1 billion CFUs to about 975 billion CFUs, or about 100 billion CFUs to about 975 billion CFUs per gram of probiotic composition. In some embodiments, the probiotic composition comprises at least about 100 million CFUs, at least about 500 million CFUs, at least about 1 billion CFUs, or at least about 100 billion CFUs per gram of probiotic composition. In some embodiments, the probiotic composition comprises about 100 million, 200 million, 300 million, 400 million, 500 million, 600 million, 700 million, 800 million, 900 million, 1 billion, 10 billion, 20 billion, 30 billion, 40 billion, 50 billion, 60 billion, 70 billion, 80 billion 90 billion, 100 billion, 200 billion, 300 billion, 400 billion, 500 billion, 550 billion, or 600 billion, 650 billion, 700 billion, 750 billion, 800 billion, 850 billion, 900 billion, 950 billion, or 1 trillion CFUs per gram of probiotic composition. In some embodiments, the probiotic composition is provided in an amount of about 100 billion, 125 billion, 150 billion, 175 billion, 200 billion, 225 billion, 250 billion, 275 billion, 300 billion, 325 billion, 350 billion, 375 billion, 400 billion, 425 billion, 450 billion, 475 billion, 500 billion, 525 billion, 550 billion, 575 billion, 600 billion, 625 billion, 650 billion, 675 billion, 700 billion, 725 billion, 750 billion, 775 billion, 800 billion, 825 billion, 850 billion, 875 billion, 900 billion, 925 billion, 950 billion, or 975 billion CFUs per gram of probiotic composition. In some embodiments, the probiotic composition comprises about 900 billion, about 950 billion, about 1.0 trillion, about 1.25 trillion, about 1.5 trillion, about 1.75 trillion, about 2.0 trillion, about 2.25 trillion, about 2.50 trillion, about 2.75 trillion, about 3.0 trillion, about 3.25 trillion, about 3.5 trillion, about 3.75 trillion, about 4.0 trillion, about 4.25 trillion, about 4.50 trillion, about 4.75 trillion, about 5.0 trillion, about 5.25 trillion, about 5.5 trillion, about 5.75 trillion, about 6.0 trillion, about 6.25 trillion, about 6.50 trillion, about 6.75 trillion, about 7.0 trillion, about 7.25 trillion, about 7.5 trillion, about 7.75 trillion, about 8.0 trillion, about 8.25 trillion, about 8.50 trillion, about 8.75 trillion, or about 9.0 trillion CFUs per gram of probiotic composition. In some embodiments, the probiotic composition comprises between about 1 trillion CFUs to about 2 trillion CFUs/g of probiotic composition. In some embodiments, the probiotic composition comprises about 1.25 trillion CFUs to about 1.50 trillion CFUs/g of probiotic composition. In some embodiments, the probiotic composition comprises about 1.3 trillion CFUs/g of probiotic composition.

In an illustrative embodiment, the probiotic composition comprises Streptococcus thermophilus, Lactobacillus delbrueckii subs. bulgaricus, and Lactobacillus rhamnosus, wherein the probiotic composition comprises about 1.25 trillion CFUs per gram of probiotic composition.

In an illustrative embodiment, the probiotic composition comprises Streptococcus thermophilus, Lactobacillus delbrueckii subs. bulgaricus, Lactobacillus rhamnosus, and Lactococcus lactis.

In an illustrative embodiment, the probiotic composition comprises Streptococcus thermophilus, Lactobacillus delbrueckii subs. bulgaricus, Lactobacillus rhamnosus, and Lactobacillus acidophilus.

In an illustrative embodiment, the probiotic composition comprises Streptococcus thermophilus, Lactobacillus delbrueckii subs. bulgaricus, Lactobacillus rhamnosus, Lactobacillus acidophilus and Lactoccocus lactis.

In another illustrative aspect, the probiotic composition comprises Streptococcus thermophilus, Lactobacillus delbrueckii subs. bulgaricus, and Lactobacillus rhamnosus, wherein the Streptococcus thermophilus is present in an amount of about 100 million CFUs to about 1 trillion CFUs/g of probiotic composition, the Lactobacillus delbrueckii subs. bulgaricus is present in an amount of about 100 billion CFUs to about 1 trillion CFUs/g of probiotic composition, and the Lactobacillus rhamnosus is present in an amount of about 100 billion CFUs to about 1 trillion CFUs/g of probiotic composition CFUs per gram of probiotic composition. In some embodiments, the composition further comprises a Lactococcus lactis present in an amount of about 100 million CFUs to about 1 trillion CFUs/g of probiotic composition.

In some embodiments, the probiotic composition is substantially free of contaminates. The following Table 1 provides an exemplary microbiological data obtained for the prepared probiotic composition.

TABLE 1
Parameters analyzed on all final batches of probiotic composition

| Microbiological Data: | Exemplary Norms |
|---|---|
| Viable Lactic acid bacteria | ≥6.9 × 10¹² |
| Lactobacillus delbrueckii subs. bulgaricus, | CFU/g* at the time |
| Lactobacillus rhamnosus | of manufacture, at |
| ISO 7889/IDF 117: 2005; ISO 9232/IDF 146: 2005; | the end of a 2-year |
| ISO 29981/IDF 220: 2010; IDF Bulletin 411: 2007 | shelf-life about 800 |
| | billion CFUs. |
| Yeast and molds | <10/g |
| ISO 6611/IDF 094: 2006 | |
| Enterobacteriaceae | Absent/g |
| ISO 4831: 2006 ISO 4832: 2006 | |
| coag. pos. Staphylococcus aureus | Absent/g |
| ISO 6888-1: 2000; ISO 6888-3: 2005 | |
| Salmonella sp. | Absent/25 g |
| ISO 6785/IDF 093: 2007 | |

### Methods of Preventing or Treating a Viral Infection

In some embodiments, the method comprises providing a probiotic composition to a subject. In some embodiments, the method comprises treating a subject suspected of having a viral infection. In some embodiments, the method comprises reducing the viral load of subject. In some embodiments, the method comprises preventing a subject from contracting a viral infection. In some embodiments, the method comprises reducing the viral infection rate of a subject. In some embodiments, the viral infection is caused by a virus capable of causing a viral respiratory infection. In some embodiments, a method of reducing a symptom of COVID-19 is provided. In some embodiments, a method of preventing a SARS-CoV-2 viral infection rate is provided. In some embodiments, a method of treating a subject suspected of having a viral infection is disclosed.

In some embodiments, the method comprises administering the probiotic composition as an orally administered mucosal vaccine. In some embodiments, the method comprises a subject orally ingesting a probiotic composition to help digestion, prevent gastrointestinal infection, support immune system health, modulate endocrine system functions such as hormonal balance, and to maintain optimal intestinal microbiota equilibrium, especially where there is heavy metal bioaccumulation, during and after antibiotic treatment, or radiation and chemotherapies.

In some embodiments, the method comprises promoting health in a subject. In some embodiments, the method comprises providing an effective amount of a probiotic composition comprising Streptococcus, Lactobacillus, and Lactococcus. In some embodiments, the method comprises providing an effective amount of a probiotic composition comprising Streptococcus and Lactobacillus. In some embodiments, the promotion of health in a subject includes improved digestion, ameliorating a bacterial infection, ameliorating a viral infection, and/or improving immune health.

In some embodiments, the method comprises administering a probiotic composition to a subject to treat a symptom of a viral respiratory infection, wherein the infection is caused, in whole or in part by, coronaviruses, rhinoviruses, respiratory syncytial viruses, influenza viruses, picornaviruses, alphaviruses, flaviviruses, or retroviruses. In some embodiments, the coronavirus is selected from SARS-CoV-2, MERS-CoV, or SARS, mutated forms of these coronaviruses, or a combination thereof. In some embodiments, the coronavirus is SARS-CoV-2.

In an illustrative embodiment, a probiotic is provided to a subject that utilizes bacteria to boost the subject's immune response to a viral infection. In some embodiments, a method for reducing incidence and/or for treatment of a SARS-CoV-2 virus infection in a subject is provided. In some embodiments, the method comprises providing or administering an effective amount of a probiotic bacteria composition. In an illustrative embodiment, the probiotic composition comprises Streptococcus, Lactobacillus, and Lactococcus.

In some embodiments, the method comprises providing the probiotic composition in a form formulated to be digested. In some embodiments, the probiotic bacteria composition is provided in a formula for oral administration. In some embodiments, wherein the probiotic composition is taken orally by a subject, the probiotic composition may be provided in a capsule, a nutrition supplement such as a chewy vitamin, an ingestible liquid, or an edible food such as yogurt. In some embodiments, the probiotic composition further comprises vitamins, protein, fiber, fruit, syrup, honey, gelatin, dairy, sugar, sweeteners, or the like to increase the nutritional value or increase the palatability of the probiotic composition including taste and/or texture.

In an illustrative embodiment, the probiotic composition may be provided in a capsule, wherein the probiotic composition is in a lyophilized form, for example freeze-dried. For example, the probiotic composition may be provided in a 400 mg veggie capsule. The 400 mg capsule may comprise between about 500 million CFUs to about 500 billion CFUs, about 125 million CFUs to 550 million CFUs, about 500 million CFUs to about 1 trillion CFUs, or about 1 billion CFUs to about 2 trillion CFUs of probiotic composition. The probiotic composition is configured to remain active for at least 12 months when stored in a cool dry place between about 4° C. to 25° C. The probiotic composition is configured to remain active for at least 24 months when stored in a cool dry place at about 12° C.

In some embodiments, wherein the probiotic composition is provided as a capsule, the capsule comprises less than about 10% moisture, less than about 9% moisture, less than about 8% moisture, less than about 7% moisture, less than about 6% moisture, less than about 5% moisture, less than about 4% moisture, less than about 3% moisture, less than about 2% moisture, or less than about 1% moisture.

In some embodiments, the method comprises providing an effective amount of a probiotic composition to a subject to treat a viral infection, the effective amount ranges from about 500 billion CFUs to about 975 billion CFUs per gram of probiotic composition. In some embodiments, the effective amount ranges from 500 billion CFUs to about 1.25 trillion CFUs. In some embodiments, the effective amount of the probiotic composition is a total of about 1.25 trillion CFUs provided as 500 billion CFU doses over a period of time such as a day.

In some embodiments, the method comprises providing an effective amount the probiotic composition to prevent a SARS-CoV-2 viral infection, the effective amount ranges from about 125 billion CFUs to about 550 billion CFUs per gram of probiotic composition.

In some embodiments, the probiotic composition is provided as a dose. In some embodiments, the dose is an amount of CFUs. For example, a dose of the probiotic composition to treat a viral infection comprises providing a dose comprising between about 500 billion CFUs to about 975 billion CFUs. For another example, a dose of probiotic composition to prevent a viral infection comprises providing a dose of probiotic composition comprising about 125 billion CFUs to about 500 billion CFUs. In some embodiments, the effective amount may be provided in a single dose or in a multiple doses over the course of the period such as a day or week. In some embodiments, the dose is an amount in grams. For example, a dose of probiotic composition to treat a viral infection comprises providing a subject a dose of about 0.10 grams to about 10 grams of the probiotic composition.

In some embodiments, the method of treating a viral infection in a subject comprises providing a subject with the probiotic composition for at least two days, at least three days, at least four days, at least five days, at least six days, or at least seven days. In some embodiments, the method comprises providing the probiotic composition to treat a subject with a viral infection for about one week, about two weeks, or about three weeks. In some embodiments, the dose is provided daily, every two days, every three days, every four days, every five days, every six days, or once a week. Alternatively, in some embodiments, a dose of probiotic composition to prevent a viral infection comprises providing a subject with the probiotic composition for at least a day to the remainder of the subject's life. In some embodiments, the probiotic composition may be provided for at least a week, at least two weeks, at least three weeks, at least a month, at least two months, or until the conditions become less opportune for catching the virus such as the ending of “flu season.”

In some embodiments, the dose may be provided once a day, twice a day, three times a day, four times a day, or at least five times a day. In some embodiments, the dose is provided once daily, up to twice daily, up to three times daily, or up to four times daily. In some embodiments, the dose is taken with other food.

In an illustrative embodiment, the method comprises providing a capsule of probiotic composition to a subject comprising between about 125 million CFUs to about 550 million CFUs or about 500 million CFUs to about 125 billion CFUs per capsule, and wherein the subject ingests the probiotic at least once a day, at least twice a day, or at least three times a day. In some embodiments, the subject may ingest the probiotic composition up to three times a day.

In some embodiments, the capsule appears to be a yellow to light brown powder with a lactic acid taste. In an illustrative embodiment, the probiotic composition, in capsule form, comprises between about 21 g to about 25 g/100 g of protein, about 3.6 g to about 4.0 g/100g of carbohydrates, wherein the carbohydrates are mainly sugars, about 2.1 g to 2.5 g/100g of Lactose, and about 0.6 g to about 1.8 g/100 g of fat. In this embodiment, the energy value is around 160 kcal/100g; 561 KJ/100 g.

In an illustrative embodiment, a method of preventing a subject from being infected by the SARS-CoV-2 virus comprises administering a probiotic composition comprising Streptococcus thermophilus, Lactobacillus delbrueckii subs. bulgaricus, and Lactobacillus rhamnosus, wherein the probiotic composition comprises about 1.25 trillion CFUs/g of probiotic composition. In some embodiments, a method to boost the immune system and keep a viral load low in a subject, comprises administering a probiotic composition to a subject comprising Streptococcus thermophilus, Lactobacillus delbrueckii subs. bulgaricus, and Lactobacillus rhamnosus, wherein the probiotic composition comprises about 1.25 trillion CFUs/g of probiotic composition.

### Kits

In another aspect, a kit is provided. The kit may include any of the probiotic compositions as described herein. In some embodiments, the kit includes one or more containers comprising the probiotic composition according to the invention. In some embodiments, the kit may additionally comprise flavoring, vitamins, fruit, sugar, sweeteners, food coloring, gelatin, or herbs.

In some embodiments, the probiotic composition is provided in a kit comprising aluminum foil bags comprising the probiotic composition under nitrogen. The probiotic composition may be provided in about 1,000 mg, about 2,000 mg, about 3,000 mg, about 4,000 mg, or about 5,000 mg per aluminum foil bag.

In some embodiments, the kit further comprises packaging to safely transport the probiotic composition. In some embodiments, the kit includes information about the contents of the probiotic composition. Further, the kit may comprise written materials, for example a user manual or answers to frequently asked questions.

The following numbered embodiments are also contemplated and are within the scope of the disclosure.

Clause 1. A probiotic composition comprising at least one probiotic strain selected from a group consisting of Streptococcus, Lactobacillus, and Lactococcus.

Clause 2. The probiotic composition of clause 1, wherein the composition further comprises a second probiotic strain selected from the group consisting of Streptococcus, Lactobacillus, and Lactococcus.

Clause 3. The probiotic composition of clause 2, wherein the composition further comprises a third probiotic strain selected from the group consisting of Streptococcus, Lactobacillus, and Lactococcus.

Clause 4. The probiotic composition of clause 3, wherein the composition further comprises a fourth probiotic strain selected from the group consisting of Streptococcus, Lactobacillus, and Lactococcus.

Clause 5. The probiotic composition of clause 4, wherein the composition further comprises a fifth probiotic strain selected from the group consisting of Streptococcus, Lactobacillus, and Lactococcus.

Clause 6. The probiotic composition of clause 5, wherein the composition further comprises a sixth probiotic strain selected from the group consisting of Streptococcus, Lactobacillus, and Lactococcus.

Clause 7. The probiotic composition of any of clauses 1-6, wherein the Streptococcus comprises Streptococcus salivarius thermophilus

Clause 8. The probiotic composition of any of clauses 1-6, wherein the Streptococcus is Streptococcus salivarius thermophilus.

Clause 9. The probiotic composition of any of clause 1-6, wherein the Lactobacillus is selected from the group consisting of Lactobacillus acidophilus, Lactobacillus rhamnosus, and Lactobacillus delbrueckii.

Clause 10. The probiotic composition of any of clauses 1-6, wherein the Lactococcus comprises Lactococcus lactis.

Clause 11. The probiotic composition of any of clauses 1-6, wherein the Lactococcus comprises Lactococcus lactis.

Clause 12. The probiotic composition of any of clauses 1-6, wherein the probiotic composition comprises Streptococcus salivarius thermophilus.

Clause 13. The probiotic composition of any of clauses 1-6, wherein the probiotic composition comprises Lactococcus lactis.

Clause 14. The probiotic composition of any of clauses 1-6, wherein the probiotic composition comprises a Lactobacillus is selected from the group consisting of Lactobacillus acidophilus, Lactobacillus rhamnosus, Lactobacillus delbrueckii, or a combination thereof.

Clause 15. The probiotic composition of clause 12, further comprising at least one strain of Lactococcus.

Clause 16. The probiotic composition of clause 15, wherein the Lactococcus is Lactococcus lactis.

Clause 17. The probiotic composition of any of clauses 12 or 13, further comprising at least one strain of Lactobacillus.

Clause 18. The probiotic composition of clause 17, wherein the Lactobacillus is selected from Lactobacillus acidophilus, Lactobacillus rhamnosus, or Lactobacillus delbrueckii.

Clause 19. The probiotic composition of clause 18, wherein the Lactobacillus is Lactobacillus acidophilus.

Clause 20. The probiotic composition of clause 18, wherein the Lactobacillus comprises a mixture of Lactobacillus acidophilus, Lactobacillus delbrueckii, and Lactobacillus rhamnosus.

Clause 21. The probiotic composition of clause 18, wherein the Lactobacillus is Lactobacillus rhamnosus.

Clause 22. The probiotic composition of clause 18, wherein the Lactobacillus is Lactobacillus delbrueckii.

Clause 23. The probiotic composition of clause 18, wherein the Lactobacillus is a mixture of Lactobacillus delbrueckii and Lactobacillus rhamnosus.

Clause 24. The probiotic composition of clause 18, wherein the Lactobacillus comprises a mixture of Lactobacillus acidophilus and Lactobacillus rhamnosus.

Clause 25. The probiotic composition of clause 17 further comprising a second strain of Lactobacillus.

Clause 26. The probiotic composition of clause 25, wherein the second strain of Lactobacillus is selected from Lactobacillus acidophilus, Lactobacillus rhamnosus, or Lactobacillus delbrueckii.

Clause 27. The probiotic composition of clause 25 further comprising a third strain of Lactobacillus.

Clause 28. The probiotic composition of clause 27, wherein the third the second strain of Lactobacillus is selected from Lactobacillus acidophilus, Lactobacillus rhamnosus.

Clause 29. The probiotic composition of clause 25 further comprising a fourth strain of Lactobacillus.

Clause 30. The probiotic composition of clause 29, wherein the third the second strain of Lactobacillus is selected from Lactobacillus acidophilus, Lactobacillus rhamnosus, or Lactobacillus delbrueckii.

Clause 31. The probiotic composition of any of clauses 1-30, wherein the probiotic composition has about 100 million to about 2.0 trillion CFUs per gram (CFUs/g) of probiotic composition.

Clause 32. The probiotic composition of any of clauses 1-30, wherein the probiotic composition has about 100 million to about 1 billion CFUs/g of probiotic composition.

Clause 33. The probiotic composition of any of clauses 1-30, wherein the probiotic composition has about 500 million to about 1 billion CFUs/g of probiotic composition.

Clause 34. The probiotic composition of any of clauses 1-30, wherein the probiotic composition has about 550 million to about 1.5 billion CFUs/g of probiotic composition.

Clause 35. The probiotic composition of any of clauses 1-30, wherein the probiotic composition has about 1 billion to about 1 trillion CFUs/g of probiotic composition.

Clause 36. The probiotic composition of any of clauses 1-30, wherein the probiotic composition has about 50 billion to about 1 trillion CFUs/g of probiotic composition.

Clause 37. The probiotic composition of any of clauses 1-30, wherein the probiotic composition has about 100 billion to about 900 billion CFUs/g of probiotic composition.

Clause 38. The probiotic composition of any of clauses 1-30, wherein the probiotic composition has about 500 billion to about 1.5 trillion CFUs/g of probiotic composition.

Clause 39. The probiotic composition of any of clauses 1-30, wherein the probiotic composition has about 550 billion to about 1.25 billion CFUs/g of probiotic composition.

Clause 40. The probiotic composition of any of clauses 1-30, wherein the probiotic composition has about 1 trillion to about 9 trillion CFUs/g of probiotic composition.

Clause 41. The probiotic composition of any of clauses 1-30, wherein the probiotic composition has about 800 billion to about 3 trillion CFUs/g of probiotic composition.

Clause 42. The probiotic composition of any of clauses 1-30, wherein the probiotic composition has about 1.25 trillion to about 1.5 trillion CFUs/g of probiotic composition.

Clause 43. The probiotic composition of any of clauses 1-30, wherein the probiotic composition has about 1.3 trillion to about 7 trillion CFUs/g of probiotic composition.

Clause 44. The probiotic composition of any of clauses 1-30, wherein the probiotic composition has about 800 billion, about 1 trillion, about 1.25 trillion, about 1.3 trillion, or about 1.75 trillion CFUs/g of probiotic composition.

Clause 45. The probiotic composition of any of clauses 1-30, wherein the probiotic composition has about 1.0×10¹³ to about 7.0×10¹⁵ CFUs/g of probiotic composition.

Clause 46. A probiotic composition comprising Streptococcus salivarius thermophilus.

Clause 47. The probiotic composition of clause 46, wherein the probiotic composition further comprises Lactococcus.

Clause 48. The probiotic composition of clause 47, wherein the Lactococcus is Lactococcus lactis.

Clause 49. The probiotic composition of any of clauses 46-48, wherein the probiotic composition further comprises a Lactobacillus.

Clause 50. The probiotic composition of clause 49, wherein the probiotic composition comprises a second Lactobacillus.

Clause 51. The probiotic composition of clause 50, wherein the probiotic composition comprises a third Lactobacillus.

Clause 52. The probiotic composition of clause 51, wherein the probiotic composition comprises a fourth Lactobacillus.

Clause 53. The probiotic composition of clause 52, wherein the probiotic composition comprises a fifth Lactobacillus.

Clause 54. The probiotic composition of any of clauses 49-53, wherein the Lactobacillus is selected from Lactobacillus acidophilus, Lactobacillus rhamnosus, or Lactobacillus delbrueckii.

Clause 55. A probiotic composition comprising Streptococcus salivarius thermophilus and Lactobacillus.

Clause 56. The probiotic composition of clause 55, wherein the Lactobacillus comprises Lactobacillus acidophilus.

Clause 57. The probiotic composition of clause 55, wherein the Lactobacillus comprises a mixture of Lactobacillus acidophilus, Lactobacillus delbrueckii, and Lactobacillus rhamnosus.

Clause 58. The probiotic composition of clause 55, wherein the Lactobacillus comprises Lactobacillus rhamnosus.

Clause 59. The probiotic composition of clause 55, wherein the Lactobacillus comprises Lactobacillus delbrueckii.

Clause 60. The probiotic composition of clause 55, wherein the Lactobacillus comprises a mixture of Lactobacillus rhamnosus and Lactobacillus delbrueckii.

Clause 61. The probiotic composition of clause 55, wherein the Lactobacillus comprises a mixture of Lactobacillus acidophilus and Lactobacillus rhamnosus.

Clause 62. The probiotic composition of clause 55, wherein the Lactobacillus comprises Lactobacillus rhamnosus and Lactobacillus delbrueckii.

Clause 63. The probiotic composition of any of clauses 9, 14, 18, 22, 26, 28, 30, 54, 59, and 62, wherein the Lactobacillus delbrueckii is Lactobacillus delbrueckii subsp. bulgaricus.

Clause 64. A probiotic composition comprising Streptococcus salivarius thermophilus and Lactococcus.

Clause 65. The probiotic composition of clause 64, wherein the Lactococcus comprises Lactococcus lactis.

Clause 66. A probiotic composition comprising Streptococcus salivarius thermophilus, Lactococcus lactis, and Lactobacillus.

Clause 67. A probiotic composition comprising Streptococcus salivarius thermophilus, Lactococcus lactis, Lactobacillus delbrueckii subsp. bulgaricus, and Lactobacillus rhamnosus.

Clause 68. The probiotic composition of clause 67 further comprising Lactobacillus acidophilus.

Clause 69. The probiotic composition of any of clauses 64-68 further comprising a mixture of Lactobacillus acidophilus and Lactobacillus rhamnosus.

Clause 70. A method of preventing a subject from catching a viral infection comprising administering any of the compositions of clauses 1-69.

Clause 71. The method of clause 70, wherein the viral infection caused by the SARS-CoV-2 virus.

Clause 72. A method of treating a subject diagnosed with COVID-19 comprising administering an effective amount of any of the compositions of clauses 1-69.

Clause 73. A method of improving the health of a subject comprising administering a composition as described in clauses 1-69.

Clause 74. The method of clause 73, wherein improving the health of a patient includes a boosted immune system, supporting gut health, improved gut health, improved limbic system function, or antibacterial and antiviral effects.

Clause 75. The method of any of clauses 70-74, wherein the probiotic composition is administered at a dose of about 550 billion CFUs to about 1.5 trillion CFUs/g of probiotic composition.

Clause 76. The method of any of clauses 70-74, wherein the probiotic composition is administered at a dose of about 800 billion CFUs to about 1.3 trillion CFUs/g of probiotic composition.

Clause 77. The method of any of clauses 75-76, wherein the probiotic composition is administered to the subject at least once a day, at least twice a day, or at least three times a day.

Clause 78. The method of any of clauses 75-76, wherein the probiotic composition is administered to a subject up to three times a day.

Clause 79. The method of any of clauses 77-78, wherein the probiotic composition is administered for up to three days, up to four days, up to five days, up to six days, up to seven days, up to eight days, up to nine days, up to ten days, up to 11 days, up to 12 days, up 13 days, or up to 14 days.

Clause 80. The method of any of clauses 77-78, wherein the probiotic composition may be administered daily for several weeks, several months, several years, or for several decades.

Clause 81. The probiotic composition or method of any of clauses 1-80 wherein the probiotic composition is formulated to be administered orally.

Clause 82. The probiotic composition or method of any of clauses 1-81, wherein the probiotic composition may be provided in a free-dried form.

Clause 83. The probiotic composition or method of any of clauses 1-82, wherein the probiotic composition is provided in a capsule form.

### EXAMPLES

The following non-limiting examples are provided to further illustrate the described embodiments and not to limit the scope of the invention.

### Example 1: Case Study of Preventing SARS-CoV-2 Infection in Human Subjects

A study, conducted in Austria from March 2020-April 2020, showed that the disclosed probiotic compositions were capable of preventing viral infection by SARS-CoV-2. Health Care Personnel, full time doctors and nurses, in contact with those infected with SARS-CoV-2 were provided a probiotic composition to reduce the risk of becoming infected with SARS-CoV-2. 150 healthy female and males with ages ranging from 30 to 75 participated in the study. The probiotic composition comprised Streptococcus salivrius thermophilus, Lactobacillus delbrueckii, and Lactobacillus rhamnosus. The Lactobacillus delbrueckii subspecies bulgaricus was used. The probiotic composition was provided in a lyophilizate form (capsule) having a CFU titer of 1.25×10¹²/grams (1.250 trillion/gram or 500 billion CFUs per capsule). The participants took a dose of 1×400 mg HPMC capsule, 3 times daily for at least 30 days. At the 30 day mark, there were no reports of any viral infection, including SARS-CoV-2 within the study group, despite working in a high-risk environment with daily exposure to COVID-19 patients and articles.

### Example 2: Case Study of Elective Use of the Probiotic Compositions by COVID-19 Patient

In early April, 2020, a 50-year-old female was brought to an emergency triage center where she was diagnosed with COVID-19. She was admitted to the Infectious Disease Ward and categorized as “high level of disease severity.” The patient stated that she had started taking probiotics and requested the ability to continue taking them while in the hospital for treatment. The medical professionals confirmed that the ingredients were not known to have counter indications to any of the COVID-19 treatments.

For three days, the patient ingested 3 capsules 3 times daily. The capsules were the same as those descried in Example 1 (i.e., 1.250 trillion/gram or 500 billion CFUs per capsule). After the three days, the patient was discharged. The patient followed up with the hospital at one week and two weeks after her discharge. During both of her visits the patient tested negative for SARS-CoV-2. On her second and final visit, it was determined that she was completely clear of COVID-19.

### Example 3: Case Study of Treating SARS-CoV-2 Infection in COVID-19 Patients

In a third study, which took place in Austria, investigators observed that elective use of probiotics to reduce symptoms and treat patients infected with SARS-CoV-2. 62 volunteers made-up of 17 females and 45 males ranging in ages between 37 and 81, were all confirmed to have COVID-19 with symptoms ranging from mild to serious. Based on symptoms, the study included 23 symptomatic outpatients and 39 symptomatic inpatients. The study specifically excluded asymptomatic individuals below 35 years of age.

Each patient was provided the same probiotic composition as described in Example 1 (i.e., a composition comprising Streptococcus salivrius thermophilus, Lactobacillus delbrueckii, and Lactobacillus rhamnosus. The Lactobacillus delbrueckii subspecies bulgaricus was used. The probiotic composition was provided in a lyophilizate form (capsule) having a CFU titer of 1.25×10¹²/grams (1.250 trillion/gram or 500 billion CFUs per capsule). Each participant took ingested the capsule 3 times daily with a meals.

The study participants were monitored weekly. After the first week, 21 patients (33.87%) tested negative for SARS-CoV-2. After the second week, 28 of the remaining 41 patients tested negative for SARS-CoV-2. Within two weeks 79.03% of the patients tested negative after two weeks. This is significant since the recovery rate for non-participants during the same period was 18.4%. During the time period of the study (April 13-27, 2020), the national recovery rate from COVID-19 in Bulgaria was 16.9%, and the worldwide recovery rate was 31.1%.

### Example 4: Case Study of Preventing and Treating a SARS-CoV-2 Infection in Patients

In a fourth study, investigators observe the effects of elective use of a probiotic composition for a first cohort of volunteer patients diagnosed with COVID-19, and a second cohort of volunteers who have not been diagnosed with COVID-19 but have daily exposure risks.

The first cohort is provided a probiotic composition comprising Streptococcus salivrius thermophilus, Lactobacillus including delbrueckii, rhamnosus, acidophilus, and Lactococcus lactis. The probiotic composition is provided in a lyophilized, capsule form where each capsule contains 500 billion CFUs. The volunteer patients took ingested a capsules three times a day for one week. The expected results is that the patients taking the probiotic composition will have a higher recovery rate in two weeks-time compared to those similarly situated but not taking the probiotic composition. It is expected that the additional strains in this probiotic composition will have an improved effect on those taking it compared to only three strains.

The second cohort is provided the same probiotic composition as the first cohort; however, each capsule contains less than 500 billion CFUs. The volunteers ingest a capsule two to three times a day with meals. The volunteers continue ingesting the probiotic composition for one month. The expected result is that the percentage of second cohort volunteers will have a lower rate of SARS-CoV-2 infection rate than those not taking the probiotic composition who are similarly situated. Further, it is expected that the second cohort of volunteers will have a lower incidence of SARS-CoV-2 infection rate compared to those only taking the three strain composition.

I claim:

1. A method of use for preventing a SARS-CoV-2 viral infection in a subject, the method comprising providing an effective amount of a probiotic composition formulated to be ingested, comprising Streptococcus salivrius thermophilus and Lactobacillus.

2. The method of claim 1, wherein the Lactobacillus is selected from the group consisting of Lactobacillus acidophilus, Lactobacillus delbrueckii, and Lactobacillus rhamnosus.

3. The method of claim 2, wherein the Lactobacillus comprises Lactobacillus delbrueckii.

4. The method of claim 3, wherein the Lactobacillus comprises Lactobacillus delbrueckii subsp. bulgaricus.

5. The method of claim 2, wherein the Lactobacillus comprises Lactobacillus rhamnosus.

6. The method of claim 4, wherein the probiotic composition further comprises Lactobacillus rhamnosus.

7. The method of claim 2, wherein the Lactobacillus comprises Lactobacillus acidophilus.

8. The method of claim 6, wherein the probiotic composition further comprises Lactobacillus acidophilus.

9. The method of claim 1, wherein the probiotic composition further comprises Lactococcus lactis.

10. The method of claim 6, wherein the probiotic composition further comprises Lactococcus lactis.

11. The method of claim 8, wherein the probiotic composition further comprises Lactococcus lactis.

12. The method of claim 1, wherein the probiotic composition comprises Streptococcus salivrius thermophilus, Lactobacillus delbrueckii subsp. bulgaricus, Lactobacillus rhamnosus, Lactobacillus acidophilus, and Lactococcus lactis.

13. The method of claim 1, wherein the effective amount is provided between about 100 million CFUs to about 3 trillion CFUs per gram of probiotic composition.

14. The method of claim 1, wherein the effective amount is provided between about 100 billion CFUs to about 3 trillion CFUs per gram of composition.

15. The method of claim 1, wherein the effective amount is provided between about 550 billion CFUs to about 1.50 trillion CFUs per gram of probiotic composition.

16. The method of claim 1, wherein the effective amount is provided between about 800 billion CFUs to about 1.3 trillion CFUs per gram of probiotic composition.

17. The method of claim 1, wherein the probiotic composition is provided in a capsule form in an amount of about 100mg to about 800 mg per capsule.

18. The method if claim 17, wherein the subject ingests the probiotic composition at least once a day up to three times a day.

19. The method of claim 1, wherein the probiotic composition further comprises vitamins, sugar, gelatin, fruit, or protein.

20. The method of claim 1, wherein the probiotic composition comprises Streptococcus salivrius thermophilus, Lactobacillus delbrueckii subsp. bulgaricus, Lactobacillus rhamnosus, Lactobacillus acidophilus, and Lactococcus lactis, and wherein the composition comprises between about 800 billion CFUs to about 1.3 CFUs/g of probiotic composition.
